(19) United States
(12) Patent Application Publication
(10) Pub. No.: US 2003/0105483 A1
(43) Pub. Date: Jun. 5, 2003

(54) Nasal packing device

(76) Inventors: John Overton Hudson, Leicester (GB); Alberto Bauer, Marbella/Malaga (ES)

Correspondence Address: Paul F Prestia, RatnerPrestia, One Westlakes Berwyn Suite 301, PO Box 980, Valley Forge, PA, 19482-0980, US

(21) Appl. No.: 10/258,556

(22) PCT Filed: May 4, 2001
(86) PCT No.: PCT/GB01/01995

(30) Foreign Application Priority Data
May 9, 2000 (GB) 0011052.8

(51) Int. Cl. A61M 29/00

(52) U.S. Cl. 606/196; 604/99.01

(57) ABSTRACT

Apparatus for arresting bleeding in a body cavity comprising a delivery means (29) and an inflatable non-elastomeric balloon (27) connectable to the delivery means (29), wherein the apparatus includes at least one pressure control means for automatically controlling inflation of the balloon (27) so that the balloon (27) can be inflated to a predetermined pressure.

The present invention relates to apparatus and methods for arresting bleeding in a body cavity. In particular, the present invention relates to a haemostatic device for controlling intra-nasal bleeding (epistaxis). The present invention also relates to the control of bleeding in many other body cavities, chambers or conduits; for example bleeding diverticula in the colon and treatment for bleeding in the oesophagus.

Various devices are used for treating bleeding inside a body cavity. Examples for the control of epistaxis include a sponge material which expands when wetted and balloon devices which apply pressure to the source of the bleeding.

Sponges consist of a polymeric material which is compressed to a relatively small size when dry, and which expand when wetted. In the process of expanding they absorb blood and fluid and apply light pressure to the source of the bleeding.

Sponges while somewhat effective in stopping bleeding have a number of disadvantages. They are hard and uncomfortable for the patient during insertion and they are not able to supply sufficient pressure to the lesion. They also do not have inherent haemostatic properties, but work by soaking up the excess blood until it forms a clot inside and around the sponge. Since the sponge is incorporated into the formed clot, and may therefore dry hard, a sponge is difficult to remove without causing re-bleeding.

A sponge also blocks the nasal passage which prohibits breathing. Although modified sponges are available which incorporate a breathing tube, the device remains inefficient and uncomfortable for the patient.

In addition, because the sponge has no actual haemostatic properties, it may have to be left in place for a considerable period (up to 48 hours). This may cause other problems such as toxic shock syndrome.

Balloon pressurised devices are also used to arrest bleeding inside body cavities. These devices consist of a balloon mounted on a tubular catheter. The balloon is inserted into a bleeding body cavity, (such as a nasal cavity), and the balloon is inflated. The balloon presses against the source of bleeding and assists the clotting of the blood in order to create haemostasis.

A considerable improvement to the devices is made when the balloon is covered by a haemostatic agent such as the device disclosed in International patent application PCT/GB98/01732, the disclosure of which is incorporated herein by reference. This patent application discloses a device suitable for treating a bleeding diverticulum or other body cavity.

One example of such a haemostatic agent is a tubular knitted fabric manufactured from carboxymethylated cellulose which is reinforced with nylon. The carboxymethylated cellulose swells and turns into a gel when in contact with blood or mucus fluid; the resultant gel acts as a strong haemostatic agent. The nylon reinforcement maintains the integrity of the fabric after the gelling takes place.

This principle is used in the “Rapid Rhino™” device made by BHK Holdings of West Bay Road, PO Box 31106, SMB Grand Caymen, Caymen Islands, British West Indies which is used to treat nose bleeding. A similar device based on the same principle is designed to treat bleeding in diverticula in the colon.

There is a technical problem with the aforementioned inflatable balloon devices because in use it is difficult to control accurately the pressure within the balloon. On the one hand, the pressure must be high enough to control the bleeding in the body cavity, whereas on the other hand if the pressure is too high then this may result in serious damage to the body cavity, ie, it may induce trauma. For example, when plugging a bleeding nose a physician must exercise extreme care so as not to damage the sensitive mucus tissue in contact with the device. Such damage may result in toxic shock syndrome. The risk of trauma may even deter certain physicians from using these devices even though their efficacy is well proven.

In known devices, the physician typically has to control the pressure in the balloon in one of two ways:

1. By inflating the balloon with a measured set volume of an inflation medium, such as air, using for example a calibrated syringe. However, this set volume method does not take into account the differing sizes of body cavities. Consequently, in the same surgical procedure a smaller than average cavity may be subjected to excessive pressure which could result in undesirable complications. Moreover, this method relies completely on the skill and diligence of the physician.

2. A second method involves inserting a safety cuff or pilot balloon, such as a second inflatable balloon, in the inflation line between the active balloon and the syringe. The safety cuff remains outside the body cavity and the physician monitors the pressure in the active balloon by manually feeling the safety cuff during inflation of the active balloon. Hence, this method relies on the experience of the physician particularly his tactile ability in feeling the correct level of pressure.

The above problems are exacerbated when inflatable balloon devices are used which are made from elastomeric materials such as silicon rubber. With an elastomeric balloon a high initial pressure is required to expand the balloon. As the balloon expands the thickness of the balloon wall decreases. This results in a decrease in force within the wall of the balloon. The effect of the balloon elasticity therefore results in a nonlinear relationship between pressure and balloon volume which increases the problems facing the physician.

Accordingly, there is need for improved methods and apparatus for treating a bleeding body cavity, in particular a bleeding nose or diverticula.

The present invention provides apparatus for arresting bleeding in a body cavity comprising a delivery means and an inflatable non-elastomeric balloon connectable to the delivery means wherein the apparatus includes at least one pressure control means for automatically controlling the inflation of the balloon so that the balloon is inflated to a predetermined pressure.

The term “automatically controlling the inflation of the balloon so that the balloon is inflated to a predetermined pressure” means that the balloon is inflated to a preselected pressure level without the need for monitoring and/or control by a physician or other users of the device.

The automatic control means solves the technical problem of ensuring that the pressure in the non-elastomeric balloon does not exceed a specific preselected value. This reduces the risk of side effects such as trauma and toxic shock syndrome. Moreover, as the method is not reliant on the skill and expertise of a particular physician the apparatus will therefore be more appealing for use by a range of users. The methods of the invention should result in improved patient compliance compared to alternative surgical procedures.

Preferably, the balloon has a soft pliable wall made from a non-elastomeric polymeric material.

It is well known to a skilled person in the art that all plastic polymers are elastic to some extent in the strict definition of the word, that is, they obey Hooke's Law and have the ability to return to their original shape after being deformed. However, it is the extent to which the polymers can be deformed which distinguishes non-elastomeric polymeric materials from elastomeric polymeric materials.

By the term “elastomeric polymeric material” we include the meaning of a polymeric material which at room temperature can be stretched to at least twice its original length and upon immediate release of the stress will quickly return to approximately its original length. Examples of elastomeric polymeric materials include rubber and silicon rubber.

By the term “non-elastomeric polymeric material” we include polymeric materials, such as nylon, which, although flexible, do not fall within the functional definition given above for elastomers.

Preferably, the balloon has a fixed volume which ensures that the pressure in the balloon is independent of the volume of the balloon. The fixed volume non-elastomeric polymeric balloon of the device of the invention ensures that adverse effects associated with wall elasticity of known elastomeric balloons are eliminated or at least substantially mitigated, as all of or most of the pressure within the balloon is directly applied to the wall of the body cavity.

Preferably, the diameter, length and volume of the balloon is designed to be slightly greater than that of the largest cavity likely to be plugged during a particular surgical procedure. More preferably, the balloon has a diameter of between 2 mm and 20 mm, a length of between 3 mm and 20 mm and a volume of between 0.004 ml³ and 5 ml³ for plugging a diverticula and a diameter of between 10 mm and 75 mm, a length of between 5 mm and 100 mm and a volume of between 0.5 ml³ and 450 ml³ for plugging a bleeding nose. Balloon devices for other body cavities such as the colon, trachea or oesophagus may be even larger.

According to a first embodiment, the pressure control means comprises an electrical pressure transducer, well known to a person skilled in the art, which constantly monitors the pressure in the balloon using an electronic instrument. Preferably, the arrangement is directly linked to an electrical inflation device, which is programmed to increase the pressure in the balloon to the required predetermined value, and automatically hold it at that value.

According to a preferred second embodiment, the pressure monitoring means comprises a valve that is operable to prevent further inflation of the balloon when the balloon is inflated to the predetermined pressure. More preferably, the valve is a pressure relief valve which is pre-set at the required pressure value and will vent pressure medium when balloon pressure reaches its pre-set value. Most preferably, the valve comprises a tubular member having an outlet in the side wall, a valve cover releasably sealing the outlet and moveable between a sealing position and an open position, and a resilient biasing means, such as a spring, for biasing the valve cover towards the sealing position so that the valve cover moves to the open position when the balloon is inflated above the predetermined pressure. Since the inflation medium is usually air, venting of excess pressure to the atmosphere is most convenient.

Silicone rubber which is used in some elastomeric balloons is permeable to air. Such silicone balloons cannot be inflated with air if inflation is to be sustained. If sustained inflation must be maintained a silicone balloon must be inflated with a liquid medium such as water or a saline solution.

If air is used as the inflation medium, then the non-elastomeric balloon used in the apparatus of the present invention must not be permeable to air. A suitable material is polyvinyl chloride (PVC), but other suitable materials may be used.

Although the relationship between volume and pressure is not linear when using an elastic inflation medium such as air, because the pressure control means is only dependent on the pressure within the balloon this non-linear behaviour of an elastic inflation medium does not affect the operation of the apparatus of the present invention.

By increasing or decreasing the force of the resilient biasing means it is possible to adjust the desirable preset pressure value in the balloon in a quick and cost effective manner. Preferably, the resilient biasing means comprises a spring load pin. The pressure value at which the valve cover will move to the open position thereby allowing the balloon to deflate is dependent on the tension of the spring and the cross-sectional area of the valve opening. Since the valve cover is only held in place by the spring, the apparatus is practically fail safe. Such valves are well known to a person skilled in the art.

In use, a user may use a syringe or a bladder type hand pump which has a larger capacity than that required to inflate the balloon to the predetermined pressure. The syringe is slowly operated to its maximum but the pressure relief valve will vent when the predetermined pressure value is reached. Hence, the balloon is inflated to the required pressure in a simple operation, without any skill or independent pressure control by the user.

Conveniently, a restriction means is provided in the inflation line distal to the pressure relief valve so that inflation is not carried out too quickly, that is, faster than the venting capacity of the relief valve.

According to a third embodiment, the pressure control means comprises a separate portable pressurised container that is charged with a pressurised inflation medium, such as compressed air. The pressurised inflation medium is charged to a specific value so that when it is connected to the delivery means, the inflatable balloon is charged to a predetermined pressure. It will be apparent to a skilled person in the art that there must be sufficient pressurised inflation medium in the container to counterbalance the pressure drop that will occur by filling the balloon.

In a particular preferred arrangement the pressure control means is operable to permit automatic controlled inflation of the balloon to at least two predetermined pressures. A particular advantage of such an arrangement is that this allows the balloon to be inflated to a relatively high initial pressure following insertion to attain immediate haemostasis. After this effect has been achieved, typically after 20 to 30 minutes, the pressure in the balloon can be reduced to mitigate possible side-effects such as trauma due to prolonged residence of the balloon in the body cavity.

Most preferably, this type of dual pressure inflation arrangement comprises two pressure relief valves, as mentioned hereinbefore, namely a first pressure relief valve that is operable to prevent further inflation of the balloon when the balloon is inflated to the predetermined pressure (up to 25 KPa for treating epistaxis); and a second pressure relief valve for reducing the pressure in the inflated balloon to a lower predetermined pressure (between 4 to 12 KPa for treating epistaxis). This type of arrangement further includes a switching means for independently switching between each of the valves to permit the balloon to be automatically inflated to different predetermined pressures.

Preferably, the switching means is a standard change over valve which may be located either on the input or exhaust side of the pressure relief valves. In use, the change over valve should initially be set so that the high pressure relief valve is in line with the balloon. After the balloon has been inflated, for example with air, for approximately 20 minutes the change over valve can be moved so that the low pressure relief valve is in line with the balloon to allow the balloon to deflate to a lower predetermined pressure.

Alternatively, the dual pressure inflation arrangement may contain a single pressure relief valve which is adapted to permit selective inflation/deflation of the balloon. For example, the tubular member of the pressure relief valve as mentioned hereinbefore may comprise two outlets each of which are releasably sealed by a valve cover. Each valve cover may be biased towards the closed position for example by two separate spring load pin arrangements, as described hereinbefore. Each of the spring loaded pins may have different tensions ie a different spring rate, and/or each of the valve openings/valve covers may have different cross-sectional areas, so that each cover opens at a different predetermined pressure. In this arrangement, the switching means permits selective communication between the balloon, inflation means and independently each outlet of the pressure relief valve.

In both of the dual pressure inflation arrangements including the pressure relief valves, the switching means may comprise removable valve caps sealing the valve outlets. In use, the balloon can be inflated/deflated to the desired predetermined pressure by simply alternately removing and replacing the valve caps.

Preferably, the delivery means for inserting the balloon into a body cavity is a catheter which includes an inflation line. Suitable catheter devices, particularly those for inserting the balloon into a diverticulum are disclosed in International patent application no. PCT/GB98/01732.

Preferably, the pressure control means is located in the inflation line of the delivery means so that in use it remains outside the patient's body, thereby increasing patient compliance. More preferably, the inflation line includes a restriction distal to the pressure relief valve to ensure that the balloon is not inflated too quickly, in particular so that the balloon is not inflated faster than the venting capacity of the relief valve. This provides the advantage of providing a further additional safety feature to ensure that the balloon is not over inflated.

Preferably, the apparatus includes a non-return valve such as a luer slip valve proximal to the restriction. Preferably, the luer slip valve is opened by the tip of the inflation device ie the syringe to allow the balloon to be inflated and the luer slip valve closes on removal of the inflation device to ensure that the inflated balloon does not deflate completely. It will be appreciated that the inflation device ie the syringe could also be used to deflate an inflated balloon by withdrawing the barrel of the syringe. Alternatively, the luer slip valve may be operable to permit deflation of an inflated balloon.

Preferably, the balloon is releasably connected to the delivery means and is associated with an agent that retards or prevents bleeding.

The term “an agent that retards or prevents bleeding” includes any haemostatic agent that is capable of arresting, stemming or preventing bleeding by means other than inducing tissue growth alone. In other words, it is not tissue growth alone which is responsible for retarding or preventing bleeding. It will of course be appreciated that the haemostatic agent may have the beneficial property of inducing tissue growth in addition to its retardation or prevention of bleeding property.

Preferably, the haemostatic agent is a bioactive compound or composition which causes vasoconstriction and/or blood coagulation.

Examples of preferred haemostatic agents that retard or prevent bleeding include oxidised cellulose, such as Tabotamp™ sold by Johnson and Johnson, calcium alginate, gelatine or collagen. A particularly preferred agent is carboxymethylated cellulose which can be purchased from Courtaulds Special Fibres, PO Box 111, 101 Lockhurst Lane, Coventry, England, CV6 5RS. Combinations of different agents may be used within the scope of the invention.

Preferably, the haemostatic agent that retards or prevents bleeding is provided in the form of a net or knitted, especially a weft knitted, textile material that envelopes the balloon. More preferably, the net or knitted textile material is fixed to the balloon and/or has a roughened surface to promote growth of fibrous tissue around the outer surface of the balloon thereby anchoring it to the interior wall of the body cavity.

Alternatively, the haemostatic agent that retards or prevents bleeding is provided in the form of a flexible film that coats the outer surface of the balloon. Preferably, the flexible film has a roughened surface to promote tissue growth.

However, it will be appreciated that if the haemostatic agent which prevents or retards bleeding is either in itself or in conjunction with the pressure exerted by the balloon relatively fast-acting, the balloon can be fixed to the delivery means and can simply be pushed inside a bleeding body cavity, such as a bleeding nose, until the bleeding has been prevented or retarded and then removed. Preferably, the body cavity comprises a bleeding nose.

The invention will be further described with reference to the following non-limiting examples and drawings wherein:

### DESCRIPTION OF THE DRAWINGS

FIG. 1 is a side view, partially cross sectioned, of a typical luer slip valve.

FIG. 2 is a plan view of the end of the valve in FIG. 1 taken along the line A-A.

FIG. 3 is the luer slip valve of FIG. 1 incorporated into a safety cuff or pilot balloon.

FIG. 4 is a plan view of the pilot balloon and valve shown in FIG. 3 taken along the line B-B.

FIG. 5 is a typical pressure relief valve.

FIG. 6 is the first embodiment of the invention with the combination luer slip valve, pressure relief valve, the restriction, the pilot balloon and inflation line.

FIG. 7 is another embodiment of the invention with two different pressure relief valves, and a sealing cap on the most proximal, and lowest pressure valve.

FIG. 8 is the pressure regulating valve system attached to a haemostatic fabric covered balloon device intended for the treatment of epistaxis.

1. Standard Inflation Port

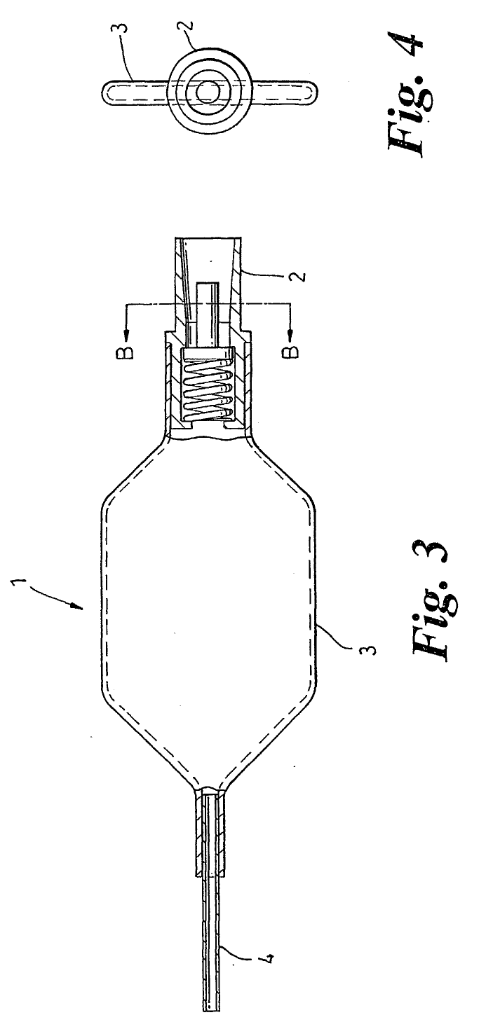

There is shown in FIGS. 3 and 4 a typical inflation port arrangement (1) used in a known balloon inflation device suitable for treating a bleeding body cavity. The device (1) consists of a luer slip valve (2), a pilot balloon (3) or safety cuff (3) and an inflation tube (4).

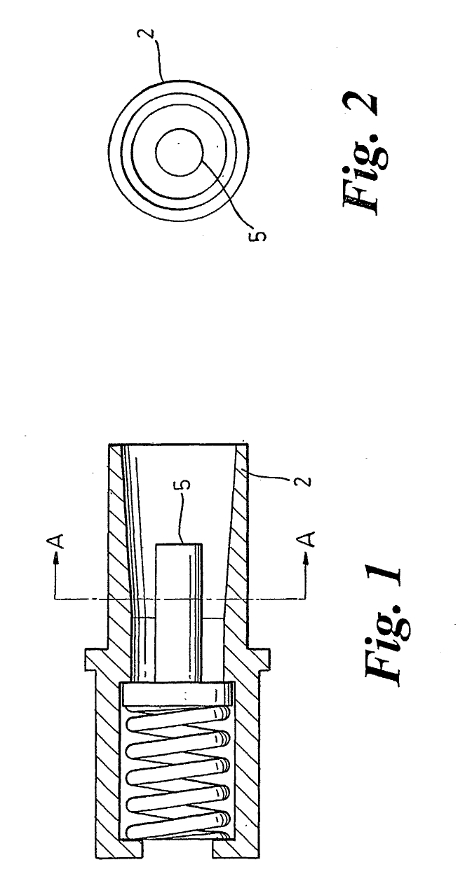

As shown in FIGS. 1 and 2 the luer slip valve (2) includes a port (5) that opens upon insertion of a tip of a syringe and automatically closes when the syringe is removed. Such an arrangement allows a balloon (not shown) to be inflated with an inflation medium and to remain inflated upon removal of the syringe.

It will be appreciated by a person skilled in the art that a bladder type hand pump fitted with a luer type inflation nozzle or a connector which is fitted to a syringe may be used instead of a syringe.

2. Pressure Relief Valve

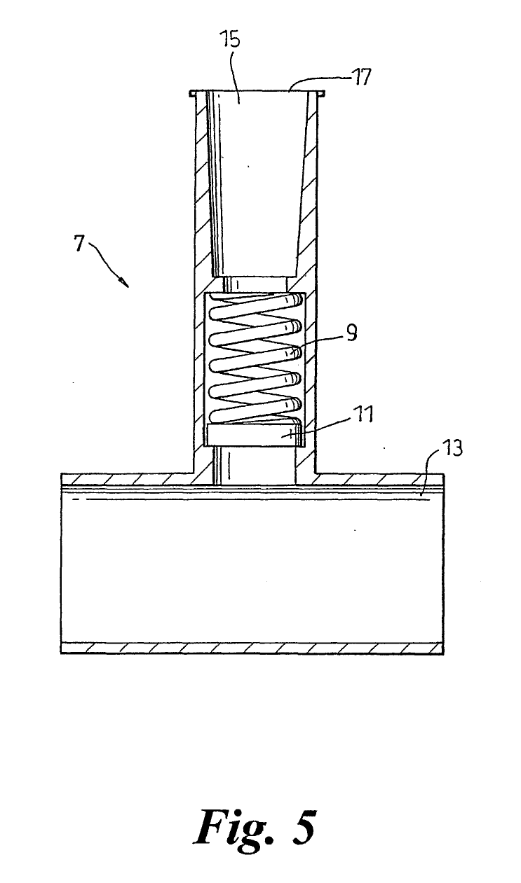

A typical pressure relief valve (7) is shown in FIG. 5. The pressure relief valve comprises a spring (9) which biases a sealing gasket (11) in towards a closed position against the pressure generated by the inflation medium in the main chamber (13). When the pressure in the main chamber (13) exerts a force on the sealing gasket (11) which exceeds the force exerted by the spring (9) biasing the sealing gasket (11) towards the closed position, the sealing gasket moves to an open position which allows the inflation medium to vent through the release vent (15). When the pressure of the inflation medium in the chamber (13) equals the force of the spring (9) exerted on the sealing gasket (11), the sealing gasket (11) will move from the open to the closed position. Thus the pressure relief valve allows a maximum predetermined pressure to be maintained in the chamber (13). The maximum predetermined pressure may be varied by changing the force exerted by the spring (9) on the sealing gasket (11) and/or by increasing/decreasing the cross-sectional area of the vent/sealing gasket (11).

Preferably, the release vent (15) is in the form of a female luer fitting so that the exit (17) make be sealed with a male luer plug (19).

3. Single Pressure Relief Valve

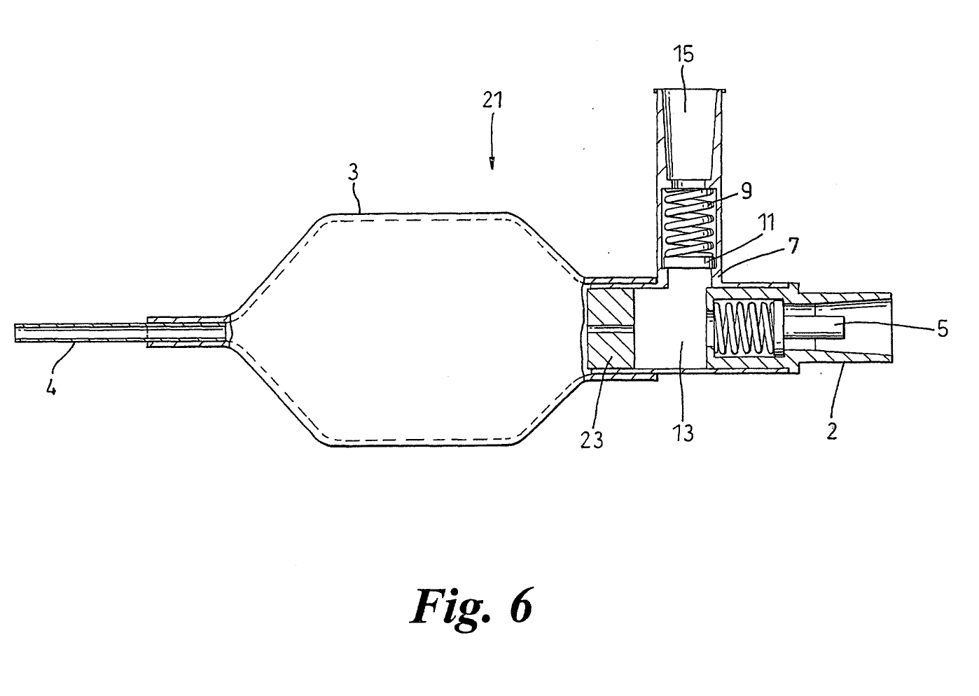

There is shown in FIG. 6 a preferred embodiment of an inflation port arrangement (21) of the apparatus of the present invention comprising a luer slip valve (2), a pressure relief valve (7), a pilot-balloon (3), an inflation line (4) and a restriction (23) distal of the pressure relief valve (7). It will be appreciated that all parts of the inflation port arrangement (21) are in fluid communication with each other and the distal end of the inflation line (4) is connectable to a non-elastomeric inflatable balloon.

The luer slip valve (2) allows air or another inflation medium to be introduced into the inflation tube (4) via a syringe, thereby inflating the non-elastomeric balloon. The pressure relief valve (7) will allow the inflation medium to vent from the system at a predetermined pressure as described hereinbefore. Hence, this inflation port arrangement (21) allows a user to inflate the balloon to a maximum pre-set pressure and to maintain the inflated balloon at that pressure (ie up to 25 KPa for treating epistaxis).

The restriction (23) ensures that the pressure of the inflation medium ie air in the inflation tube (4) does not rise above the predetermined maximum value as it prevents the inflation medium from being forced into the inflation tube (4) faster than the rate at which the vent (15) can vent excess pressure in the chamber (13).

4. Dual Pressure Relief Valve

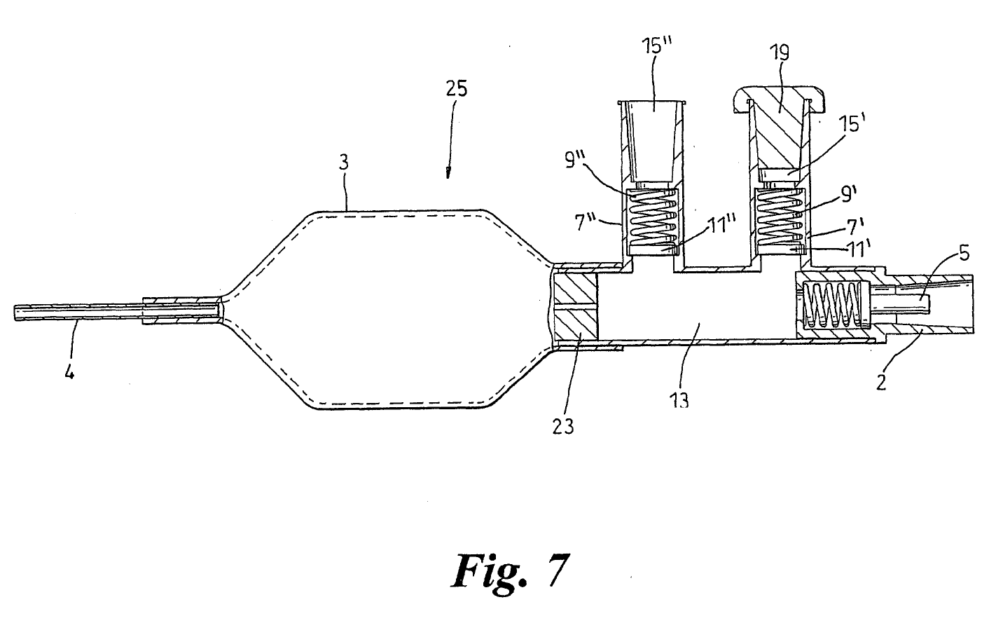

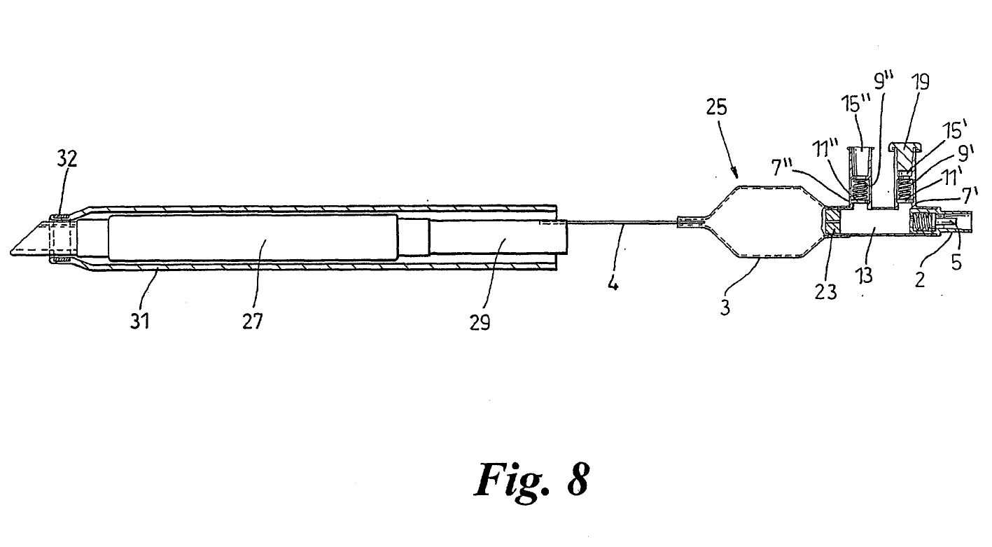

There is shown in FIG. 7 an alternative preferred embodiment of an inflation port arrangement (25) of the apparatus of the present invention comprising two pressure relief valves (7′, 7″), a luer slip valve (2), a pilot balloon (3), an inflation line (4) and a restriction (23) distal of the pressure relief valves (7′, 7″).

In FIG. 7 and FIG. 8, the parts described hereinbefore are indicated by the same reference numerals.

The two pressure relief valves (7′, 7″) comprise a pressure relief valve (7′) which is adapted to vent at a lower predetermined pressure than the other pressure relief valve (7″). As mentioned above, this may be achieved by using springs (9′, 9″) having different tensions and sealing gaskets (11′, 11″) having different cross-sectional areas. This arrangement allows an inflatable balloon (27) to be inflated to two different predetermined pressures.

FIG. 8 illustrates the inflation port arrangement (25) in combination with an inflatable non-elastomeric balloon (27) mounted on a delivery catheter (29) which is in fluid communication with the inflation line (4). Typically, the inflation line is about 40-50 mm long when the device is used for treating epistaxis.

The balloon (27) includes a hemostatic fabric shroud (31) secured to the distal tip of the balloon (27) by a fabric ring clamp (32).

For treating epistaxis, the balloon (27) and hemostatic fabric shroud (31) are inserted into a bleeding nostril. The vent (15′) of the low pressure relief valve (7′) is initially capped with a male luer cap (19) to prevent the inflation medium ie air from venting through this valve (7′). The second higher pressure relief valve (7″) initially has its vent (15″) open.

A syringe containing air is inserted into the inflation port (5) of the luer slip valve (2) and air introduced into the apparatus. The balloon (27) inflates to the higher preset pressure limit, ie between 12 to 25 KPa, as determined by the higher pressure relief valve (7″) and remains inflated at this pressure. This high pressure relief valve (7″) may be configured so that the pressure in the balloon is slightly higher than normal blood pressure. This enables rapid haematosis to be attained.

After an initial haematosis has been achieved the balloon may be deflated to the lower preset pressure, ie between 4 to 12 KPa, by removing the male luer cap (19) from the vent (15′) of the low pressure relief valve (7′). This allows the healing nasal cavity to stabilise, it is more comfortable for the patient, and it is less likely to cause medical complications ie deformation of the nasal cavity.

It will be appreciated by a person skilled in the art that although the illustrations in this description have referred to standard luer fittings any suitable seals, fasteners, vents, vent caps and connectors may be used.

Furthermore, any number of pressure relief valves could be used to allow an inflation balloon(s) to be inflated to a number of different pre-set pressure values. Each preset pressure value could be chosen by selectively closing the vents of the pressure relief valves by luer lock caps or some other form of sealing connector.

It will also be appreciated by a skilled person that the pressure control means, ie the pressure relief valves, may be bypassed if each vent is closed by a luer plug. In this case the balloon may be inflated to a non preset value, which may be desirable in certain surgical procedures.

Alternatively, a valve having a vent sealed with a plug but without a spring loaded seal may be included in the apparatus of the present invention. Removal of the plug from this type of valve will prevent the balloon from being inflated, and if the balloon is already inflated, will cause it to deflate. This type of valve will allow the balloon to be deflated in an emergency situation even if a syringe is not readily to hand.

It is important to match the pressure controlled inflation system with a non-elastomeric, fixed volume balloon. Provided the volumes of the balloon is bigger than the cavity which is being treated then the pressure in the system is the same as the pressure applied to the bleeding cavity. This is in contrast to using a balloon made from an elastomeric material where some of the pressure in the balloon is utilised in overcoming the forces within the balloon material itself. Consequently, with an elastomeric balloon there is no direct control of the actual force applied to the bleeding cavity.

The devices incorporated in this invention are typically low pressure devices and will work at pressures up to approximately 25 KPa (Kilo pascals). A typical dual pressure device may have the high pressure set between 12 and 25 KPa and the low pressure set between 4 and 12 KPa. However, the principles of the invention should not be restricted to such low pressure devices.

1. Apparatus for arresting bleeding in a body cavity comprising a delivery means and an inflatable non-elastomeric balloon connectable to the delivery means, wherein the apparatus includes at least one pressure control means for automatically controlling inflation of the balloon so that the balloon can be inflated to a predetermined pressure.

2. Apparatus as claimed in claim 1 wherein the balloon has a fixed volume.

3. Apparatus as claimed in claim 1 or 2 wherein the balloon is constructed from a non-elastomer polymeric material.

4. Apparatus as claimed in claim 3 wherein the non-elastomer polymeric material is PVC.

5. Apparatus as claimed in any one of the preceding claims wherein the pressure control means is operable to permit automatic controlled inflation of the balloon to at least two predetermined pressures.

6. Apparatus as claimed in any one of the preceding claims wherein the pressure control means comprises an electrical pressure transducer.

7. Apparatus as claimed in any one of claims 1 to 5 wherein the pressure control means includes a valve that is operable to prevent further inflation of the balloon when the balloon is inflated to the predetermined pressure.

8. Apparatus as claimed in claim 7 wherein the valve is a pressure relief valve which prevents further inflation of the balloon when the balloon is inflated to the predetermined pressure.

9. Apparatus as claimed in claim 8 wherein the pressure relief valve comprises a tubular member having an outlet in a side wall, a valve cover releasably sealing the outlet and moveable between a sealing position and an open position, and resilient biasing means for biasing the valve cover towards the sealing position so that the valve cover moves to the open position when the balloon is inflated above the predetermined pressure.

10. Apparatus as claimed in claim 5 wherein the apparatus includes at least two valves as defined in any one of claims 7 to 9, each of said valves being operable to permit automatic controlled inflation of the inflatable balloon to a different predetermined pressure, and further including switching means for switching between each of said valves to permit the balloon to be automatically inflated to each of the different predetermined pressures.

11. Apparatus as claimed in claim 10 when dependent on claim 9 wherein the switching means includes removable valve caps sealing the valve outlets.

12. Apparatus as claimed in claim 11 wherein the at least two valves form a unitary valve member.

13. Apparatus as claimed in claim 10 wherein the at least two valves form a unitary valve member.

14. Apparatus as claimed in any one of the preceding claims wherein the delivery means includes an inflation line that is connectable at a distal end to the inflatable balloon and the pressure control means is located in the inflation line or at the proximal end of the inflation line.

15. Apparatus as claimed in claim 14 when dependent on claims 7 to 13 wherein the inflation line includes a restriction means distal to the valve for controlling the rate of inflation of the balloon.

16. Apparatus as claimed in claim 1 wherein the pressure control means comprises a container including a pressurised fluid and the container is connectable to the delivery means to permit inflation of the inflatable balloon to the predetermined pressure.

17. Apparatus as claimed in claim 16 wherein the container is fitted with a pressure relief valve to prevent the balloon from being inflated above the predetermined pressure.

18. Apparatus as claimed in any one of the preceding claims further including a non-return valve to prevent deflation of the balloon.

19. Apparatus as claimed in claim 18 wherein the non-return valve is a luer slip valve.

20. Apparatus as claimed in any one of the preceding claims wherein the inflatable balloon is associated with an agent that retards or prevents bleeding.

21. Apparatus as claimed in claim 20 wherein the agent that retards or prevents bleeding is selected from one or more of oxidised cellulose, carboxymethylated cellulose, calcium alginate, gelatine or collagen.

22. Apparatus as claimed in claim 20 or 21 wherein the agent that retards or prevents bleeding is provided in the form of a net or a knitted textile material that envelopes the inflatable balloon or in the form of a flexible film that coats the outer surface of the balloon.

23. Apparatus as claimed in claim 22 wherein the net or knitted textile material is fixed to the balloon.

24. Apparatus as claimed in any one of the preceding claims wherein the balloon is not permeable to air.

25. Apparatus as claimed in any preceding claim wherein the delivery means is a catheter.

26. Apparatus as claimed in any one of the preceding claims wherein the inflatable balloon is releasably connected to the delivery means.

27. Apparatus as claimed in any one of the preceding claims wherein the apparatus is adapted to permit the pressure control means to remain external to the body of a patient.

28. Apparatus as claimed in any one of the preceding claims wherein the apparatus includes a single pressure control means.

29. Use of the apparatus as defined in any one of claims 1 to 28 for preventing bleeding of a nose.

30. Apparatus for arresting bleeding in a body cavity substantially as described herein, and preferably with reference to one or more of the accompanying drawings.

31. A method of arresting bleeding in a body cavity comprising providing apparatus as defined in any one of claims 1 to 28 and 33, inserting the inflatable balloon in the body cavity, and inflating the inflatable balloon to a predetermined pressure.
